(19) United States
(12) Patent Application Publication
(10) Pub. No.: US 2010/0197694 A1
(43) Pub. Date: Aug. 5, 2010

(54) Compositions and methods for treatment of diseases and conditions with increased vascular permeability

(76) Inventor: Gerald Horn, Deerfield, IL (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 12/798,929

(22) Filed: Apr. 14, 2010

Related U.S. Application Data
(63) Continuation-in-part of application No. 12/460,970, filed on Jul. 27, 2009.
(60) Provisional application No. 61/137,714, filed on Aug. 1, 2008.
(60) Provisional application No. 61/192,777, filed on Sep. 22, 2008.
(60) Provisional application No. 61/203,120, filed on Dec. 18, 2008.
(60) Provisional application No. 61/207,481, filed on Feb. 12, 2009.
(60) Provisional application No. 61/287,518, filed on Dec. 17, 2009.

(51) Int. Cl. A61K 31/498; A61K 31/4164; A61K 31/165

(52) U.S. Cl. 514/249; 514/396; 514/617

(57) ABSTRACT

The invention provides compositions and methods for treating diseases and conditions through reducing vascular permeability, selectively inhibiting VEGF-induced postcapillary venular leakage, and/or selectively reducing spread of viral and/or bacterial pathogens. The provided compositions and methods utilize low concentrations of selective α-2 adrenergic receptor agonists having a binding affinity of 300 fold or greater for α-2 over α-1 adrenergic receptors. The compositions preferably comprise brimonidine and/or dexmedetomidine.

### CROSS-REFERENCE TO RELATED APPLICATIONS

This application is a continuation-in-part of U.S. patent application Ser. No. 12/460,970, filed Jul. 27, 2009, which claims a priority of U.S. Provisional Application Ser. Nos. 61/137,714, filed on Aug. 1, 2008; 61/192,777, filed on Sep. 22, 2008; 61/203,120, filed on Dec. 18, 2008; and 61/207, 481 filed on Feb. 12, 2009. This application also claims a priority of U.S. Provisional Application Ser. No. 61/287,518, filed on Dec. 17, 2009. The contents of the above-mentioned application are hereby incorporated by reference in their entirety.

### BACKGROUND OF THE INVENTION

Vascular Endothelial Growth Factor (VEGF) is an important molecule produced by cells which stimulates the growth of new blood vessels. Among other functions, VEGF maintains vascular integrity and is therefore a critical regulator responsible for maintaining the proper functioning of the blood vessels, including vascular permeability.

Vascular permeability may be divided into three basic categories: basal (i.e., normal physiologic functions); acute (associated with various pulmonary pathologies); and chronic (associated with such conditions as, for example, tumor angiogenesis, chronic hypoxia, and others).

In many diseases and conditions (including but not limited to pulmonary diseases and conditions such as asthma, bronchiolitis, pneumonia, and others), acute vascular permeability is accompanied by an elevated level of VEGF. An elevated level of VEGF causes an inflammatory cascade, generally starting with vascular leakage and culminating with severe inflammation. Vascular leakage is primarily characterized by venular postcapillary leakage as a predominant component. This leakage involves several mechanisms and may include stripping of vascular endothelial cadherins (VE-cadherins) and formation of large endothelial gaps which leak plasma and proteins, including large proteins, such as fibrin. The leaking plasma, proteins and remaining exudative debris leak into bronchi and smaller bronchioles. There, they are exposed to clotting factors which precipitate large fibrin clots that further reduce cillary mucous clearance and add mucous plugs. The cumulative result is inspissated (i.e., thickened/trapped) “secretions.” These accumulated secretions cause collapse of alveoli, further block mucous clearance, diminish alveolar gas exchange, attract water, solutes, and debris into the clots, and are very strong chemoattractants to neutrophils, promoting a strong inflammatory reaction as well as increasing the risk of infection due to stasis and reduced clearance of organic debris in the affected area(s).

The currently available anti-VEGF agents are ineffective and potentially deleterious for treating pulmonary diseases and conditions because they inhibit multiple and/or substantially all functions of VEGF, where such functions are multifactorial and considered essential for maintenance of normal vascular integrity within the lung. Thus, these anti-VEGF agents are ill-suited to treat pulmonary diseases and conditions.

Accordingly, there is a need for new compositions and methods that would inhibit harmful effects of elevated VEGF on vascular permeability without increasing the risk of untoward imbalance and/or deterioration of essential vascular integrity and homeostasis.

### SUMMARY OF THE PRESENT INVENTION

The present invention provides compositions and methods to treat and/or prevent diseases and conditions through reducing vascular permeability, selectively inhibiting VEGF-induced postcapillary venular leakage, and/or selectively reducing spread of viral and/or bacterial pathogens utilizing low concentrations of selective α-2 adrenergic receptor agonists which selectively constrict smaller blood vessels.

In some embodiments of the invention, the selective α-2 adrenergic receptor agonists have binding affinities (KO for α-2 over α-1 receptors of 300:1 or greater. In preferred embodiments of the invention, the selective α-2 adrenergic receptor agonists have Kᵢ for α-2 over α-1 receptors of 500:1 or greater. In more preferred embodiments of the invention, the selective α-2 adrenergic receptor agonists have Kᵢ for α-2 over α-1 receptors of 700:1 or greater. In more preferred embodiments of the invention, the selective α-2 adrenergic receptor agonists have Kᵢ for α-2 over α-1 receptors of 1000:1 or greater. In even more preferred embodiments of the invention, the selective α-2 adrenergic receptor agonists have Kᵢ for α-2 over α-1 receptors of 1500:1 or greater.

In preferred embodiments of the invention, the selective α-2 adrenergic receptor agonists have binding affinities (Kᵢ) of 100 fold or greater for α-2b and/or α-2c receptors over α-2a receptors.

In preferred embodiments of the invention, the selective α-2 adrenergic receptor agonist is selected from the group consisting of brimonidine, dexmedetomidine, guanfacine, 4-NEMD, and mixtures of these compounds.

In preferred embodiments of the invention, concentrations of the selective α-2 adrenergic receptor agonists are from about 0.0001% to about 0.05%; more preferably, from about 0.001% to about 0.05%; even more preferably, from about 0.01% to about 0.025%; and even more preferably, from about 0.01% to about 0.02% weight by volume of the composition.

Thus, in one embodiment, the invention provides a composition comprising a selective α-2 adrenergic receptor agonist having a binding affinity of 300 fold or greater for α-2 over α-1 adrenergic receptors, or a pharmaceutically acceptable salt thereof, wherein said α-2 adrenergic receptor agonist is present at a concentration from between about 0.001% to about 0.05% weight by volume.

In preferred embodiments, the compositions and methods of the invention may comprise potassium chloride and/or calcium chloride.

Preferably, the concentration of potassium chloride is between about 10 mM and 80 mM, most preferably about 20 mM to 40 mM, and the concentration of calcium chloride is between about 0.05 mM and about 2 mM, most preferably about 1 mM.

In preferred embodiments, a pH of the composition of the invention is between about 4.0 and about 6.5.

In one embodiment, the invention provides a composition comprising between about 0.01% to about 0.05% weight by volume of brimonidine, further comprising from between about 0.05 to about 2 mM of calcium chloride, from between about 10 mM to about 80 mM of potassium chloride and wherein pH of said composition is between about 4.0 and about 6.5.

In one embodiment, the invention provides a composition comprising between about 0.01% to about 0.025% weight by volume of dexmedetomidine, further comprising from between about 0.05 to about 2 mM of calcium chloride, from between about 10 mM to about 80 mM of potassium chloride and wherein pH of said composition is between about 4.0 and about 6.5.

In some aspects, the compositions and methods of the invention further comprise other therapeutic agents, including bronchodilators and/or antibiotics.

In preferred embodiments, the bronchodilators may include, but are not limited to, selective and/or non-selective β-2 adrenergic receptor agonists, anticholinergics, and theophylline.

The invention also provides methods of treating and/or preventing a disease or condition comprising administering to a patient in need thereof a therapeutically effective amount of the compositions of the invention.

Without wishing to be bound to any particular theory, in preferred embodiments, the compositions and methods of the invention result in reduced vascular permeability believed to be caused by postcapillary venular constriction induced by the inventive compositions and methods. Thus, the compositions and methods of the invention reduce the large VEGF-induced postcapillary venular gaps and related vascular permeability increase, resulting in selective inhibition of the acute vascular permeability increase and related inflammatory and hypoxic sequelae caused by elevated levels of VEGF. This postcapillary venular constriction is believed to be increased in hypoxic conditions typical of pulmonary pathology associated with VEGF increase.

The compositions and methods of the present invention are believed to be capable of reducing vascular permeability, selectively inhibiting VEGF-induced postcapillary venular leakage, and/or selectively reducing spread of viral and/or bacterial pathogens.

Accordingly, in one embodiment, the invention provides methods of inducing a selective vasoconstriction of smaller blood vessels, such as microvessels, capillaries, and/or postcapillary venules relative to larger blood vessels, such as arteries and/or proximal arterioles. This selective vasoconstriction of smaller blood vessels allows for such effects while decreasing and/or eliminating ischemia risk. Unlike the present invention, α-1 agonists induce constriction of large and small vessels, for example causing constriction of the pulmonary artery. Therefore, α-1 agonists may considerably increase ischemia and secondarily inflammation. They are also direct agonist constrictors of bronchiole muscularis, which is equally or more damaging, since they cause direct bronchiole constriction, which is a highly deleterious and dangerous effect in respiratory compromised patients.

In accordance with the present invention, reduction of vascular permeability may reduce spread of viral and/or bacterial pathogens into surrounding lung parenchyma and may therefore reduce morbidity. The inventive compositions and methods may also selectively inhibit VEGF-induced postcapillary venular leakage,

In some aspects, the invention provides methods and compositions for treatment of pulmonary diseases and conditions that reduce or eliminate the need for steroids currently required in conventional treatments of pulmonary diseases and conditions. The steroid use can also decrease vascular permeability; however it usually requires many hours or even days for this decrease to be pronounced, with the maximum effect in many days or even weeks. This long time frame renders steroids not sufficiently active for the treatment of acute exacerbation of pulmonary conditions.

In some aspects, the compositions of the invention may be administered via aerosolized delivery and/or inhalation delivery.

### BRIEF DESCRIPTION OF THE FIGURES

FIG. 1 is a graphical representation of the factors causing airway obstruction in asthma patients;

FIG. 2 is a graphical representation of the effects of alveolar pneumonia;

FIG. 3 is a graphical representation of the effects of brimonidine and saline on central airway resistance in rats.

### DETAILED DESCRIPTION OF THE INVENTION

### Definitions

The term “selective α-2 adrenergic receptor agonists” encompasses all α-2 adrenergic receptor agonists which have a binding affinity of 300 fold or greater for α-2 over α-1 adrenergic receptors. The term also encompasses pharmaceutically acceptable salts, esters, prodrugs, and other derivatives of selective α-2 adrenergic receptor agonists.

The term “low concentrations” refers to concentrations from between about 0.0001% to about 0.05%; more preferably, from about 0.001% to about 0.05%; even more preferably, from about 0.01% to about 0.025%; and even more preferably, from about 0.01% to about 0.02% weight by volume of the composition.

The term “brimonidine” encompasses, without limitation, brimonidine salts and other derivatives, and specifically includes, but is not limited to, brimonidine tartrate, 5-bromo-6-(2-imidazolin-2-ylamino)quinoxaline D-tartrate, Alphagan™, and UK14304.

The term “dexmedetomidine” encompasses, without limitation, dexmedetomidine salts and other derivatives.

As used herein, the term “composition” is intended to encompass a product comprising the specified ingredients in the specified amounts, as well as any product which results, directly or indirectly, from a combination of the specified ingredients in the specified amounts.

The terms “treating” and “treatment” refer to reversing, alleviating, inhibiting, or slowing the progress of the disease, disorder, or condition to which such terms apply, or one or more symptoms of such disease, disorder, or condition.

The terms “preventing” and “prevention” refer to prophylactic use to reduce the likelihood of a disease, disorder, or condition to which such term applies, or one or more symptoms of such disease, disorder, or condition. It is not necessary to achieve a 100% likelihood of prevention; it is sufficient to achieve at least a partial effect of reducing the risk of acquiring such disease, disorder, or condition.

### Embodiments of the Invention

The invention provides compositions and methods to treat and/or prevent diseases and conditions through reducing vascular permeability, selectively inhibiting VEGF-induced postcapillary venular leakage, and/or selectively reducing spread of viral and/or bacterial pathogens utilizing low concentrations of selective α-2 adrenergic receptor agonists which selectively constrict smaller blood vessels and selectively inhibit VEGF.

While not wishing to be bound to any particular theory, it is believed that VEGF plays a very important role in many diseases and conditions, including pulmonary diseases and conditions. Elevated VEGF levels cause postcapillary venular permeability increase and/or release of exudate which, among other consequences, may cause bronchiole obstruction, atelectasis (i.e., collapse of lung sacs), and other deleterious effects. Conventional α-2 agonists are poorly suited for treating pulmonary diseases and conditions because they cause constriction of both smaller and larger blood vessels, thereby contributing to ischemia. The compositions and methods of the invention are able to counteract the deleterious effects of elevated VEGF levels without causing ischemia because they selectively cause constriction of smaller blood vessels (especially, postcapillary venules) while not affecting larger blood vessels.

It is believed that one of the reasons why the compositions and methods of the invention are is because they are highly selective for α-2 adrenergic receptor agonists. Many α-2 agonists are also α-1 agonists, and therefore if α-2 agonists are insufficiently selective for α-2 receptors, they may have deleterious consequences associated with stimulating α-1 adrenergic receptors, causing profound vasoconstriction of large vessels contributing to ischemia, and/or inducing bronchiole constriction via α-1 muscularis receptors. Moreover, even selective α-2 adrenergic receptor agonists, when used at conventional doses of 0.1% or higher are associated with a number of undesirable side effects, such as rebound hyperemia and secondary vasodilation. These effects may be associated with a “cross-over” stimulation of α-1 adrenergic receptors as even the relatively low α-1 receptor stimulation versus α-2 receptor stimulation for these selective α-2 agonists becomes cumulatively significant at higher concentrations and α-1 agonist effects increasingly dominate because they are so untoward and potentially dangerous in these circumstances. Accordingly, the invention utilizes selective α-2 agonists at low concentrations whereby α-2 receptor agonist activity is almost exclusively induced, and α-1 adrenergic receptors are not sufficiently stimulated to cause negative effects as described above.

The pulmonary diseases and conditions that may be treated with the compositions and methods of the present invention include, but are not limited to, asthma, persistent asthma, status asthmaticus, as well as other forms of pulmonary diseases and conditions, including Methicillin-resistant Staphylococcus aureus (MRSA), strep, pneumoccal, viral and other forms of pneumonia, certain types of pulmonary edema, respiratory syncytial virus (RSV) disease, cystic fibrosis (particularly where bronchiectasis and/or atelectasis persist), acute respiratory distress syndrome, bronchiolitis, lung transplant rejection syndrome, and acute lung injury.

### Selective α-2 Adrenergic Receptor Agonists Suitable for the Purposes of the Invention

In some embodiments of the invention, the selective α-2 adrenergic receptor agonists have binding affinities (Kᵢ) for α-2 over α-1 receptors of 300:1 or greater. In preferred embodiments of the invention, the selective α-2 adrenergic receptor agonists have Kᵢ for α-2 over α-1 receptors of 500:1 or greater. In more preferred embodiments of the invention, the selective α-2 adrenergic receptor agonists have Kᵢ for α-2 over α-1 receptors of 700:1 or greater. In more preferred embodiments of the invention, the selective α-2 adrenergic receptor agonists have Kᵢ for α-2 over α-1 receptors of 1000:1 or greater. In even more preferred embodiments of the invention, the selective α-2 adrenergic receptor agonists have Kᵢ for α-2 over α-1 receptors of 1500:1 or greater.

It is well within a skill in the art to design an assay to determine α-2/α-1 functional selectivity. As non-limiting examples, potency, activity or EC₅₀ at an α-2A receptor can be determined by assaying for inhibition of adenylate cyclase activity. Furthermore, inhibition of adenylate cyclase activity can be assayed, without limitation, in PC12 cells stably expressing an α-2A receptor such as a human α-2A receptor. As further non-limiting examples, potency, activity or EC₅₀ at an α-1A receptor can be determined by assaying for intracellular calcium. Intracellular calcium can be assayed, without limitation, in HEK293 cells stably expressing an α-1A receptor, such as a bovine α-1A receptor.

The particularly preferred adrenergic receptor agonists for the purposes of the present invention have higher selectivity for α-2B and/or α-2C receptors, as compared to α-2A receptors within the lung. In preferred embodiments of the invention, the selective α-2 adrenergic receptor agonists have binding affinities (Kᵢ) of 100 fold or greater for α-2b and/or α-2c receptors over α-2a receptors. While not wishing to be bound to any specific theory, it is believed that α-2b receptors have the predominant peripheral vascular vasoconstrictive role in arterioles and venules. At the same time, α-2a receptors are predominantly found in the central nervous system, and therefore, α-2a specific agonists have a lesser role in causing direct vascular constriction and reduction of vascular permeability.

In cases of severe pulmonary compromise secondary to acute vascular permeability, including those that may be VEGF-induced, in addition to the α-2 and preferential α-2b agonist-induced microvessel terminal arteriolar and postcapillary venular constriction, a further advantage of the inventive compositions and methods may be activation of central nervous system (CNS) α-2a receptors. Activation of CNS α-2a receptors has been shown to have sedative effects, which may be beneficial in cases of bronchial constriction where anxiety and emotional stress are often contributing factors in cases refractory to treatment, and CNS α-2a receptors are also thought to be involved in a mechanism inducing bronchiole dilation.

In preferred embodiments of the invention, concentrations of selective α-2 adrenergic receptor agonists are from about 0.0001% to about 0.05%; more preferably, from about 0.001% to about 0.05%; even more preferably, from about 0.01% to about 0.025%; and even more preferably, from about 0.01% to about 0.02% weight by volume of the composition.

Any selective α-2 adrenergic receptor agonist with Kᵢ for α-2 over α-1 receptors of 300:1 or greater may be suitable for the purposes of the present invention. In preferred embodiments, Kᵢ for α-2 over α-1 receptors is 500:1 or greater, more preferably, 700:1 or greater, even more preferably 1000:1 or greater, and even more preferably 1500:1 or greater. In other preferred embodiments, the inventive compositions are more selective for α-2b receptors versus α-2a receptors.

Compositions and methods of the inventions encompass all isomeric forms of the described α-2 adrenergic receptor agonists, their racemic mixtures, enol forms, solvated and unsolvated forms, analogs, prodrugs, derivatives, including but not limited to esters and ethers, and pharmaceutically acceptable salts, including acid addition salts. Examples of suitable acids for salt formation are hydrochloric, sulfuric, phosphoric, acetic, citric, oxalic, malonic, salicylic, malic, furmaric, succinic, ascorbic, maleic, methanesulfonic, tartaric, and other mineral carboxylic acids well known to those in the art. The salts may be prepared by contacting the free base form with a sufficient amount of the desired acid to produce a salt in the conventional manner. The free base forms may be regenerated by treating the salt with a suitable dilute aqueous base solution such as dilute aqueous hydroxide potassium carbonate, ammonia, and sodium bicarbonate. The free base forms differ from their respective salt forms somewhat in certain physical properties, such as solubility in polar solvents, but the acid salts are equivalent to their respective free base forms for purposes of the invention. (See, for example S. M. Berge, et al., “Pharmaceutical Salts,” J. Pharm. Sci., 66: 1-19 (1977) which is incorporated herein by reference).

As long as a particular isomer, salt, analog, prodrug or other derivative of a selective α-2 adrenergic receptor agonist functions as a selective α-2 agonist, it may be used for the purposes of the present invention.

When choosing a particular α-2 adrenergic receptor agonist, one may take into account various considerations including any possible side effects and other systemic reactions.

In select circumstances, it may be preferable for the active agent of the present invention to penetrate parenchymal cell membranes, in which case a higher pH, including pH of greater than 7 may be desired. In this event, solubility may be reduced and require anionic components to stabilize. Such anionic components may include peroxide and/or other solubility enhancers and/or preservatives.

In preferred embodiments of the invention, the selective α-2 adrenergic receptor is brimonidine or its salt. In a more preferred embodiment, the selective α-2 adrenergic receptor agonist is the tartrate salt of brimonidine.

### Compositions and Methods of the Invention

In one embodiment, the invention provides a composition comprising a selective α-2 adrenergic receptor agonist having a binding affinity of 300 fold or greater for α-2 over α-1 adrenergic receptors, or a pharmaceutically acceptable salt thereof, wherein said α-2 adrenergic receptor agonist is present at a concentration from between about 0.001% to about 0.05% weight by volume.

In a preferred embodiment, the selective α-2 adrenergic receptor agonist is present at a concentration below about 0.05% weight by volume, and more preferably, between about 0.001% to about 0.05% weight by volume.

In one embodiment, the selective α-2 adrenergic receptor is selected from the group consisting of brimonidine, dexmedetomidine, guanfacine, 4-NEMD, and mixtures of these compounds.

In a preferred embodiment, the composition comprises brimonidine at a concentration between about 0.001% and about 0.05% weight by volume.

In a more preferred embodiment, a pH of the composition comprising the selective α-2 adrenergic receptor agonist is between about 4.0 and about 6.5.

In another preferred embodiment, the compositions of the present invention further include potassium (i.e., K⁺). The term “potassium” includes, but is not limited to, potassium salt. In a preferred embodiment, potassium is in the form of potassium chloride (KCl) and its concentration is between about 10 mM and 50 mM, most preferably about 40 mM.

In another preferred embodiment, the compositions of the present invention further include calcium (i.e., Ca²⁺). The term “calcium” includes, but is not limited to, calcium salt. In a preferred embodiment, calcium is in the form of calcium chloride (CaCl₂) and its concentration is between about 0.05 mM and 2 mM, most preferably about 1 mM.

In preferred embodiments, a pH of the composition of the invention is between about 4.0 and about 6.5.

In some aspects, the compositions and methods of the invention further comprise other therapeutic agents, including bronchodilators and/or antibiotics.

In preferred embodiments, the bronchodilators may include, but are not limited to, β-2 adrenergic receptor agonists, anticholinergics, and theophylline.

In one embodiment, the invention provides a composition comprising between about 0.01% to about 0.05% weight by volume of brimonidine, further comprising from between about 0.05 to about 2 mM of calcium chloride, from between about 10 mM to about 80 mM of potassium chloride and wherein pH of said composition is between about 4.0 and about 6.5.

In one embodiment, the invention provides a composition comprising between about 0.01% to about 0.025% weight by volume of dexmedetomidine, further comprising from between about 0.05 to about 2 mM of calcium chloride, from between about 10 mM to about 80 mM of potassium chloride and wherein pH of said composition is between about 4.0 and about 6.5.

The compositions of the present invention are preferably formulated for a mammal, and more preferably, for a human.

The invention also provides methods of treating and/or preventing a pulmonary disease or condition comprising administering to a patient in need thereof a therapeutically effective amount of the compositions of the invention.

In preferred embodiments, the compositions and methods of the invention cause postcapillary venular constriction thus counteracting the clinically damaging increase in acute vascular permeability caused by elevated levels of VEGF.

In some embodiments, the compositions and methods of the invention selectively inhibit VEGF-induced postcapillary venular leakage.

In some embodiments, the compositions and methods of the invention selectively reduce the spread of viral and/or bacterial pathogens.

In some embodiments, the invention provides methods of inducing a selective vasoconstriction of smaller blood vessels, such as microvessels, capillaries, and/or postcapillary venules relative to larger blood vessels, such as arteries and/or arterioles. This selective vasoconstriction of smaller blood vessels allows decreasing and/or eliminating ischemia.

In some aspects, the invention provides methods and compositions for treatment of pulmonary diseases and conditions that reduce or eliminate the need for steroids currently required in conventional treatments of pulmonary diseases and conditions.

In some aspects, the compositions of the invention may be administered via aerosolized delivery and/or inhalation delivery.

### Aerosolized and Nebulized Compositions

In preferred embodiments, the compositions of the invention are aerosolized or nebulized. In one embodiment, the aerosolized or nebulized composition is formulated for treating and/or preventing a pulmonary condition. It is within a skill in the art to prepare the aerosolized compositions of the present invention.

The aerosolized or nebulized compositions of the present invention are generally delivered via an inhaler, jet nebulizer, or ultrasonic nebulizer which is able to produce aerosol particles with size of between about 1 and 10 μm.

In one embodiment, the invention provides an aerosolized composition comprising a selective α-2 adrenergic receptor agonist having a binding affinity of 300 fold or greater for α-2 over α-1 adrenergic receptors, or a pharmaceutically acceptable salt thereof, wherein said α-2 adrenergic receptor agonist is present at a concentration from between about 0.001% to about 0.05% weight by volume.

In one embodiment, the selective α-2 agonist may be formulated in about 5 ml solution of a quarter normal saline having pH between 4.5 and 6.5, preferably between 4.5 and 6.0.

In a preferred embodiment, the aerosolized or nebulized composition comprises about 0.02% weight by volume of brimonidine in about 5 ml solution, which further comprises about 0.225% weight by volume of sodium chloride, and wherein said composition has a pH between about 4.5 and about 6.5.

In some embodiments, particularly for the treatment of influenza and other pulmonary pathogen infections, the aerosolized or nebulized compositions are delivered in sufficient concentrations to create effective systemic/local tissue as well as local mucosal concentration of the drug.

In preferred embodiments, the aerosolized or nebulized compositions are delivered at sufficient concentration and in sufficient duration to create effective systemic/local tissuel as well as local mucosal concentration of the drug.

In some embodiments, the aerosolized or nebulized compositions are effective for systemic effect on the central nervous system.

In some embodiments, the aerosolized or nebulized compositions are effective for treating pulmonary disorders or conditions.

In some embodiments, the invention provides a method of treating influenza and/or a secondary lung infection comprising administering to a patient in need thereof a selective α-2 adrenergic receptor agonist, or a pharmaceutically acceptable salt thereof, wherein said selective α-2 adrenergic receptor agonist is present at a concentration below about 0.05% weight by volume.

In some embodiments, the invention provides a method of treating ambulatory asthma or upper/lower respiratory congestion by administering to a patient in need thereof a metered dose of a composition comprising 0.05% by weight of brimonidine or dexmedetomidine via an inhalant.

In some embodiments, the invention provides a method of treating influenza, status asthmaticus, or persistent severe asthma by administering to a patient in need thereof a nebulized composition comprising 0.05% by weight of brimonidine or dexmedetomidine for about 1 to 3 minutes.

In some embodiments, the secondary drug infection is a pneumococcal infection.

In a preferred embodiment, the invention provides a method of treating influenza and/or pneumococcal infection comprising administering to a patient in need thereof about 0.05% weight by volume of a selective α-2 adrenergic receptor agonist, wherein said selective α-2 adrenergic receptor agonist is nebulized. Preferably, the treatment time is about 30 min.

In general, conditions that are less acute may be treated via metered dose by inhaler, including cases of asthma, upper and lower respiratory congestion and walking pneumonia. Conditions that are more acute may require nebulized drug. Such conditions include but are not limited to persistent asthma, status asthmaticus, viral and/or bacterial pneumonia, respiratory syncytial virus disease, infant bronchiolitis, and acute lung injury.

Compositions for Oral and/or Intravenous Administration

In some embodiments, the compositions of the present invention can be included in a pharmaceutically suitable vehicle suitable for oral ingestion. Suitable pharmaceutically acceptable carriers include solid fillers or diluents and sterile aqueous or organic solutions. The active compound is present in such pharmaceutical compositions in an amount sufficient to provide the desired effect.

Pharmaceutical compositions contemplated for use in the practice of the present invention can be used in the form of a solid, a solution, an emulsion, a dispersion, a micelle, a liposome, and the like, wherein the resulting composition contains one or more of the active ingredients in admixture with an organic or inorganic carrier or excipient suitable for nasal, enteral, or parenteral applications.

The active ingredients may be combined, for example, with the usual non-toxic, pharmaceutically and physiologically acceptable carriers for tablets, pellets, capsules, troches, lozenges, aqueous or oily suspensions, dispersible powders or granules, suppositories, solutions, emulsions, suspensions, hard or soft capsules, caplets or syrups or elixirs and any other form suitable for use. The possible carriers include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents may be used.

In yet another embodiment, the compositions of the present invention may be formulated for an intravenous (IV) administration. It is within a skill in the art to formulate the compositions for an IV administration.

Diseases and Conditions to be Treated with Compositions and Methods of the Invention

The invention provides compositions and methods that may be used to treat or prevent a variety of diseases and conditions, including but not limited to pulmonary diseases and conditions. Pulmonary diseases and conditions include, but are not limited to cystic fibrosis and various other forms of pulmonary diseases and conditions, including edemas (including interstitial edema, pulmonary edema, and other edemas), vascular congestion, mucosal swelling of bronchi and bronchioles, infectious tracheobronchitis, respiratory syncytial virus (RSV) bronchitis, etc. Other pulmonary uses include treatments of increased vascular leakage/permeability that further swell the bronchiole mucosa and shrink the available lumen size of an airway. Such increases in vascular permeability occur in allergic rhinitis, common cold; influenza; asthma, exercise induced asthma, acute respiratory distress syndrome, and acute lung injury. Such conditions produce added increase in generalized inflammation and secretions with high morbidity in such conditions as pneumonia, and cystic fibrosis after secondary pneumonia, as often occurs with secondary pneumococcal and other infections. Such conditions can cause alveolar capillary increased permeability and capillary changes along the mucosal surface that swell the mucosa into the lumen. An increase in vascular permeability is known as one of the main features by which these pathogens are disseminated inside a host organism through cascade of inflammatory byproducts and other specific means of induction. In addition, the compositions and methods of the inventions can be used to treat patients after undergoing sinus surgery.

Unlike prior art pulmonary treatment for lung pathologies, in which a specific drug is applicable to a single or limited pathologies, the present invention allows for unique properties specific to α-2 agonists to be effective in the entire spectrum of respiratory conditions, from those affecting the proximal oropharynx, to the most distal lung parenchyma.

Three specific attributes are optimized by the combination of high selectivity and low concentration that results in α-2 receptor trigger without untoward α-1 receptor activation: 1) the anesthetic properties of α-2 agonists; 2) the vasoconstrictive-vascular permeability reducing properties; and 3) the CNS properties of sedation and bronchiole dilation.

By offering a spectrum of particle sizing, inhalation vehicles, and flow rates an expert in the art can provide treatment with the present invention localized to primarily the distribution best for that specific entity, ranging from oropharynx for pharyngeal inflammation (sore throat), upper respiratory tract conditions (URI); lower respiratory tract conditions (LRI); known distal distribution of bronchiole B2 receptors to achieve primarily luminal relief for congested/constricted airways; to the most distal, lung parenchyma, as with viral and infectious conditions that have primarily or secondarily reached beyond and/or far beyond the respiratory tract itself to systemic absorption.

Particle sizing is typically in the range of 1 μm to 8 μm, where the smallest size droplets reach most distal into lung parenchyma and the largest most proximal. For the present invention this is extended to about 0.01 μm to about 20 μm, allowing for a more complete range of proximal to distal droplet anatomic targeting as the present invention can utilize. A dispenser designed to allow an adjusted or multiple sized combination with a range of 0.01 μm to 50 μm, and more preferably 0.05 μm to 20 μm, and still more preferably 1 um to 10 um could be used to allow for treatment of a variety of conditions, for example proximal spectrum largest droplet size: pharyngitis+upper respiratory airway disease; midspectrum mid range droplet size: lower respiratory tract+B2 selective distribution; to distal spectrum smallest droplet size: lung parenchyma and maximal systemic absorption for CNS distribution at the other end of the spectrum.

For treatment of specific conditions with the present invention where bronchiole constriction is the primary pathophysiologic process, such as for asthma treatment, use of highly selective alpha 2 agonists, with binding affinity of 300 more higher, along with concentration of 0.001-0.05% and particle size of preferably 2-7 μm, more preferably 2.5-5.0 μm, and more preferably 2.0-3.0 μm is recommended. Aerosol should be delivered to the alveoli if delivery to the circulatory system is desired, using particle sizes in the smaller droplet of this range, provided the particle has a density similar to water, and a generally spherical shape. Particles with higher or lower density will effectively behave as bigger or smaller particles, respectively. Diseases of small airways and alveoli (e.g., asthma, emphysema, pulmonary infections, etc.) may similarly require delivery with small, typically 1-2 μm, spherical particles. Therefore, the present invention allows for and can be optimized for targeted delivery to areas of bronchiole constriction to reduce luminal congestion, as well as CNS absorption to reduce anxiety and through CNS trigger reduce bronchiolar constriction as well. A particle size of 2.5 μm combined with one of 1.5 μm or less allows for both bronchiolar areas and CNS absorption.

For treatment of pneumonia, bacterial or viral where lung parenchyma absorption is the primary pathophysiologic process, use of highly selective alpha 2 agonists, with binding affinity of 300 or higher, along with concentration of 0.001-0.059% and particle size of preferably 0.5-3 μm, more preferably 1-2.5 μm, and still more preferably 1.5 μm-2.0 μm is recommended. Utilizing higher pH, such as between 6.5 and 8.0, and more preferably 6.8 to 7.5, and still more preferably 7.0-7.25 allows for greater lipophilic cell membrane permeabilities, as occurs in cell walls of lung parenchymal tissue to still further increase such desired absorption. As solubility of alpha 2 agonists decreases with pH at or above 7.0 some solubility enhancement as known to experts in the art, such as with anionic stabilizers and/or preservatives such as peroxide based compounds may be added, however the low concentrations desired for the highly selective alpha 2 agonists per this invention minimizes this need to one of possible preference and mild enhancement of efficacy rather than necessity, one also dependent on the concentration within the range of the invention desired.

It has been further documented that in patients following sinus surgery increased access to and absorption within the sinus cavities results, where particle sizes from 1 μm to 25 μm may be effectively absorbed. This is particularly true for the maxillary sinus after maxillary sinuses after maxillary antrostomy and ethmoidectomy. Treatment with the present invention in the postoperative state can reduce swelling, inflammation, and related morbidity.

Particle size can be controlled by a variety of means, such as use of porous membranes of various pore dimensions. Further applying energy to reduce the bulk of aerosolized media may optionally be employed to enhance the percentage of smaller particle sizes as desired; pore size of the aerosolization membrane; temperature of aerosolization; extrusion velocity; ambient humidity; the concentration, surface tension, viscosity of the formulation; and vibration frequency.

Aerosol particle size can be adjusted by adjusting the size of the pores of the membrane, as discussed, for example, in U.S. Pat. No. 7,244,714.

The present invention is more fully demonstrated by reference to the accompanying drawings.

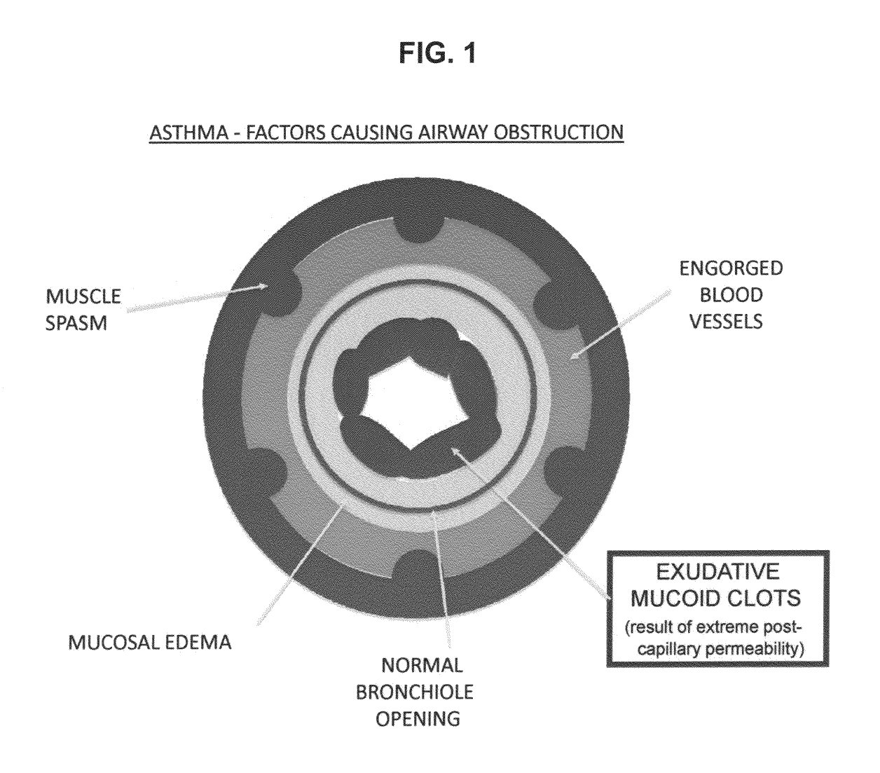

FIG. (FIG.) 1 is a graphical representation of the factors causing airway obstruction in asthma patients. As FIG. 1 demonstrates, these factors include muscle spasm, mucosal edema, engorged blood vessels, and exudative mucoid clots. These clots are a result of extreme postcapillary venular permeability believed to be increased at the site of action, and causally related to (or, at the very least contributed to) elevated VEGF levels.

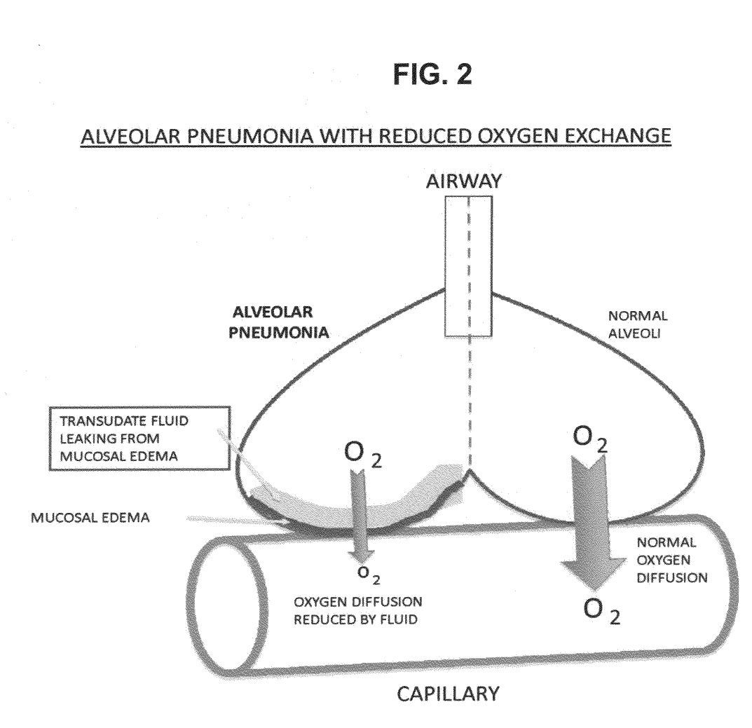

FIG. 2 is a graphical representation of the effects of alveolar pneumonia. As FIG. 2 demonstrates, in alveolar pneumonia, transudate fluid is leaking from mucosal edema, causing reduction in oxygen diffusion in the capillaries.

The following Examples are provided solely for illustrative purposes and is not meant to limit the invention in any way.

### EXAMPLE 1

### Effect of Brimonidine vs Saline on Airway Secretions in Inflamed Lungs

The purpose of this experiment was to compare the effect of brimonidine vs saline on the amount of airway secretions in inflamed lungs of rats. The experiment was designed as follows. 10 rats were administered either saline solution (6 rats) or brimonidine at 200 μg/ml (0.02%), 400 μg/ml (0.04%), and 800 μg/ml (0.08%) (4 rats).

The resistance at the first time point prior to administration of saline or brimonidine was established at 100%, establishing the baseline. The mean resistance at baseline was similar for the two treatment groups, and therefore, all the measured resistances were expressed as a % of the baseline resistance. After establishing baseline conditions, the first aerosol treatment (saline or brimonidine at 200 μg/ml) was delivered for one minute, followed by 10 minutes of monitoring. The airway resistance at the end of the 10-minute period was the first post-treatment resistance, and the airway resistance measured immediately after removal of tracheal secretions was the second post-treatment resistance. These measurements were repeated for two more aerosol treatments in each rat.

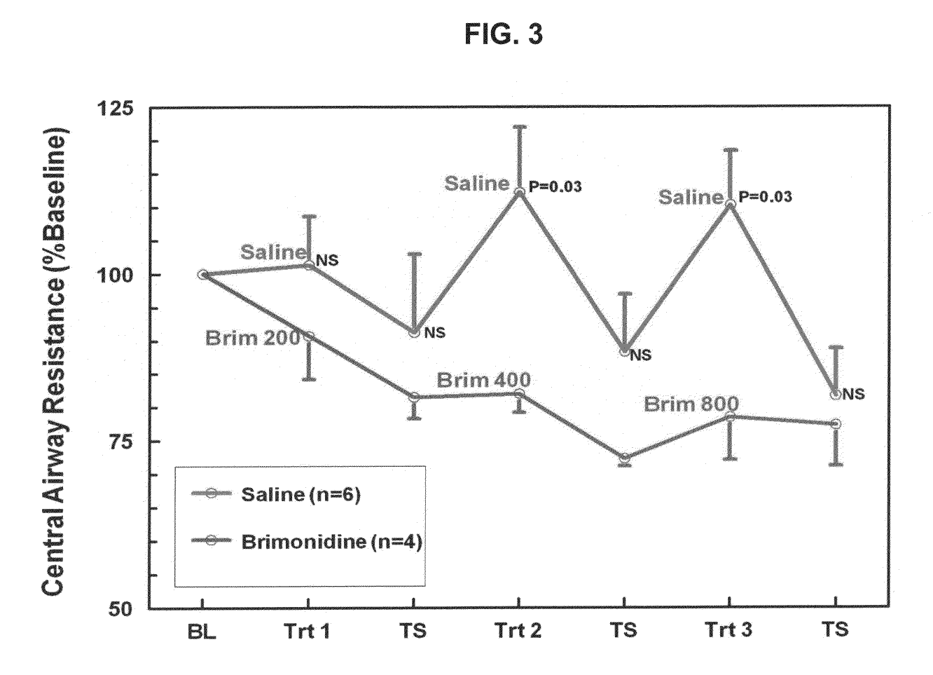

The 2 groups were compared statistically at various post-treatment time points. FIG. 3 is a plot of Central Airway Resistance (% of baseline) vs various time points. Trt 1, Trt 2, and Trt 3 denote 10-minute periods immediately after first, second, and third treatments, respectively. TS denotes a time point after tracheal secretions were removed after the treatments.

Results

In the saline-treated rats, there is an increase in resistance after 10 minutes after each aerosol treatment (i.e. at Trt 1, Trt 2, and Trt 3). However, the effect is completely reversed after tracheal suctioning (i.e., at TS points). This suggests that the increases in resistance are due to accumulating secretions. However, in the brimonidine-treated rats, there is little increase in resistance during the post-treatment monitoring period, and little change after suctioning. Statistically, the post-treatment resistances are significantly higher (P=0.03) in the saline group compared with the brimonidine group after the 400 and 800 μg/ml doses, but there is not a significant difference between groups for the post-suctioning resistances at any of the time points. Further, the final pre/post-suctioning change in resistance is significantly greater in the saline group than the brimonidine group (P=0.006).

These observations are consistent with brimonidine reducing the accumulation of airway secretions in inflamed lungs. The lack of a significant difference in post-suctioning resistances between the saline and brimonidine groups suggests that brimonidine did not alter airway mucosal edema substantially in the central airways.

### EXAMPLE 2

### Prophetic

### Effect of Brimonidine and Dexmedetomidine on Inhibition of VEGF Inflammatory Cascade

The purpose of this experiment is to test the effect of administering aerosolized brimonidine and dexmedetomidine on pulmonary function in acute respiratory viral infection.

Study Design

A parallel group design of five groups of eight rats each: virus/saline, virus/brimonidine, virus/dexmedetomidine, sham/saline, sham/brimonidine. Treatments are twice daily, beginning one day post inoculation, and ending the morning of terminal studies on day 4, 5 or 6 post inoculation.

Treatments
1) Brimonidine tartrate 0.05% aerosol, generated with ultrasonic nebulizer (12 ml solution loaded into nebulizer for each treatment), delivered into a holding chamber, and breathed spontaneously by awake rats for 5 minutes twice daily (0800 and 1800 hrs), beginning one day after viral inoculation.
2) Dexmedetomidine HCl 0.05% aerosol, generated with ultrasonic nebulizer (12 ml solution loaded into nebulizer for each treatment), delivered into a holding chamber, and breathed spontaneously by awake rats for 5 minutes twice daily (0800 and 1800 hrs), beginning one day after viral inoculation.
3) Control treatment: pH-matched saline aerosol.

A 5-minute exposure is recommended due to the lag time of filling the exposure box with aerosol after the rats have been loaded into the box

### Viral Infection

Rats will be inoculated with Parainfluenza type 1 (Sendai) virus via aerosol exposure, and housed in isolation cubicles. Control groups will be sham-inoculated with virus-free vehicle, and housed in an identical manner.

### Assessment

daily body weights;
lung function: oxygenation on room air (pulse oximetry); lung mechanics (pressure-volume curve, quasistatic elastance, dynamic elastance); airflow resistance (Newtonian resistance, respiratory system resistance, tissue damping);
lung inflammation: right lung bronchoalveolar lavage, with total leukocyte and differential leukocyte counts;
pulmonary transudate & exudates: left lung wet/dry weight ratio determined for 6 rats in each group;
formalin-fixed, paraffin-imbedded left lungs from 2 rats in each group; mid-sagittal thin sections prepared with H&E stain.

What is claimed is:

1. A composition comprising a selective α-2 adrenergic receptor agonist having a binding affinity of 300 fold or greater for α-2 over α-1 adrenergic receptors, or a pharmaceutically acceptable salt thereof, wherein said α-2 adrenergic receptor agonist is present at a concentration from between about 0.001% to about 0.05% weight by volume for use in treatment of a disease or condition with increased vascular permeability.

2. The composition of claim 1, wherein said selective α-2 adrenergic receptor agonist has a binding affinity of 700 fold or greater for α-2 over α-1 adrenergic receptors.

3. The composition of claim 1, wherein said selective α-2 adrenergic receptor agonist has a binding affinity of 1000 fold or greater for α-2 over α-1 adrenergic receptors.

4. The composition of claim 1, wherein said selective α-2 adrenergic receptor has a binding affinity of 100 fold or greater for α-2b and/or α-2c receptors over α-2a adrenergic receptors

5. The composition of claim 1, wherein said selective α-2 adrenergic receptor agonist is selected from the group consisting of brimonidine, dexmedetomidine, guanfacine, 4-NEMD, and mixtures of these compounds.

6. The composition of claim 1, wherein said composition further comprises potassium chloride.

7. The composition of claim 1, wherein said composition further comprises calcium chloride.

8. The composition of claim 1, wherein said increased vascular permeability is primarily induced by VEGF elevation.

9. A composition comprising a selective α-2 adrenergic receptor agonist having a binding affinity of 300 fold or greater for α-2 over α-1 adrenergic receptors, or a pharmaceutically acceptable salt thereof, wherein said α-2 adrenergic receptor agonist is present at a concentration from between about 0.001% to about 0.05% weight by volume for use in selectively inhibiting VEGF-induced postcapillary venular leakage.

10. The composition of claim 9, wherein said selective α-2 adrenergic receptor agonist has a binding affinity of 700 fold or greater for α-2 over α-1 adrenergic receptors.

11. The composition of claim 9, wherein said selective α-2 adrenergic receptor agonist has a binding affinity of 1000 fold or greater for α-2 over α-1 adrenergic receptors.

12. A composition comprising a selective α-2 adrenergic receptor agonist having a binding affinity of 300 fold or greater for α-2 over α-1 adrenergic receptors, or a pharmaceutically acceptable salt thereof, wherein said α-2 adrenergic receptor agonist is present at a concentration from between about 0.001% to about 0.05% weight by volume for use in selectively reducing spread of viral and/or bacterial pathogens.

13. The composition of claim 12, wherein said selective α-2 adrenergic receptor agonist has a binding affinity of 700 fold or greater for α-2 over α-1 adrenergic receptors.

14. The composition of claim 12, wherein said selective α-2 adrenergic receptor agonist has a binding affinity of 1000 fold or greater for α-2 over α-1 adrenergic receptors.
